(19) United States
(12) Patent Application Publication
(10) Pub. No.: US 2012/0208887 A1
(43) Pub. Date: Aug. 16, 2012

(54) STABLE, LIQUID, READY-TO-USE KETOPROFEN FORMULATIONS

(76) Inventors: Didier Douleau, Pyla sur Mer (FR); Julien Renard, Lambersart (FR)

(21) Appl. No.: 13/431,556

(22) Filed: Mar. 27, 2012

Related U.S. Application Data
(63) Continuation of application No. PCT/FR2010/000641, filed on Sep. 27, 2010.

(30) Foreign Application Priority Data
Sep. 28, 2009 (FR) 0904622
Sep. 27, 2010 (WO) FR2010/000641

(51) Int. Cl. A61K 31/19; A61P 13/12; A61P 29/00

(52) U.S. Cl. 514/570

(57) ABSTRACT

Stable, liquid, ready-to-use ketoprofen formulation for parenteral administration are provided. In some embodiments, the formulation comprises ketoprofen solubilized in a solely aqueous solvent comprising a buffering agent, wherein the composition has a pH of about 5.5 to about 6.5.

### CROSS REFERENCE TO RELATED APPLICATIONS

This application is a continuation of PCT/FR2010/000641, filed Sep. 27, 2010, which claims the benefit of French application No. 0904622, filed Sep. 28, 2009, the entire disclosures of which are hereby incorporated by reference.

### BACKGROUND

The present disclosure generally relates to stable, liquid, ready-to-use ketoprofen formulations, in particular for parenteral administration, particularly intravenous administration. In some embodiments, the present disclosure applies to the administration of ketoprofen by perfusion to treat postoperative pain and renal colic attacks.

The use of ketoprofen in racemate or enantiomer form as an analgesic, anti-inflammatory and antipyretic medication that is well-tolerated by humans is known. In fact, ketoprofen is one of the most active nonsteroidal anti-inflammatory drugs (NSAID). It forms part of the derivatives of propionic acid, having rapid anti-inflammatory and analgesic activity.

Ketoprofen has very low solubility in water (52 mg/L) and in acid solutions. This poor solubility is explained by the presence of an aromatic lipophilic group. Ketoprofen is more soluble in basic solutions but then degrades rapidly, demonstrating the instability of such basic solutions.

In France, a single specialty that is intravenously injectable exists, sold under the name of Profenid®. The specialty is present in the form of a lyophilizate vial containing 100 mg of ketoprofen, sodium hydroxide, glycine and citric acid. The lyophilizate must be dissolved extemporaneously in an isotonic solution of glucose or sodium chloride before patient injection. However, the ketoprofen solution thus reconstituted presents greater osmolarity than that of the plasma and causes pain when injected into the patient. Adjusting the osmolarity is not possible after its reconstitution. In addition, reconstitution of the medical solution requires additional medical equipment, such as transfer devices, perfusion lines and solvent bags. It generates time for the medical personnel and causes, during the solution reconstitution time, a delay in the pain treatment of a patient.

In order to overcome the ketoprofen solubility problems, several methods have been proposed, including the use of a co-solvent or the formation of complexes or nitrogen salts of ketoprofen. Aqueous solutions of such complexes or nitrogen salts present a pH equal to or greater than 7.

In the article Singhai. et al, Pharmazie 52:226-228 (1997), it is indicated that the solubility and stability of ketoprofen in water are increased by using the co-solvents polyethylene glycol and propylene glycol or sodium benzoate. However, polyethylene glycol is not recommended for intravenous administration due to its toxicity.

U.S. Pat. No. 5,895,789 proposes an aqueous solution for parenteral administration comprising an alkyl ammonium salt of ketoprofen, for example the L-lysine salt of ketoprofen, the solution having a pH between 7 and 7.5 and being free of preservatives, co-solvents and support substances such as glycine. However, this solution must be produced and maintained in an inert gas atmosphere that requires special facilities.

Moreover, document WO-99/52528 describes hydrosoluble ketoprofen salts obtained by reaction of ketoprofen with glucosamine, proline and/or hydroxyproline and used in injectable preparations, tablets or gels.

Forming complexes of ketoprofen with piperazine or ethylenediamine to increase ketoprofen solubility and reduce irritation during parenteral administration has also been contemplated in document WO-2006/116626. The pH of solutions containing these complexes is between 6.5 and 8.5.

### SUMMARY

The present disclosure generally relates to stable, liquid, ready-to-use ketoprofen formulations, in particular for parenteral administration, particularly intravenous administration. In some embodiments, the present disclosure applies to the administration of ketoprofen by perfusion to treat postoperative pain and renal colic attacks.

The applicant has observed that by using a solely aqueous solution comprising a buffering agent and by fixing the pH of the solution around 6, ketoprofen is solubilized without the need for the presence of a non-aqueous co-solvent. In addition, these aqueous solutions are stable over time.

Accordingly, in one embodiment, the present disclosure provides stable, liquid, ready-to-use ketoprofen compositions comprising ketoprofen solubilized in a solely aqueous solvent comprising a buffering agent, wherein the composition has a pH of about 5.5 to about 6.5.

In another embodiment, the present disclosure provides a sterile bag comprising a stable, liquid, ready-to-use ketoprofen composition comprising ketoprofen solubilized in a solely aqueous solvent comprising a buffering agent, wherein the composition has a pH of about 5.5 to about 6.5.

In yet another embodiment, the present disclosure provides a method of preparing a stable, liquid, ready-to-use ketoprofen composition comprising dissolving ketoprofen in a solely aqueous solvent comprising a buffering agent, and adjusting the pH of the composition to a value of about 5.5 to about 6.5.

The features and advantages of the present invention will be apparent to those skilled in the art. While numerous changes may be made by those skilled in the art, such changes are within the spirit of the invention.

### DESCRIPTION

The present disclosure generally relates to stable, liquid, ready-to-use ketoprofen formulations, in particular for parenteral administration, particularly intravenous administration. In some embodiments, the present disclosure applies to the administration of ketoprofen by perfusion to treat postoperative pain and renal colic attacks.

According to a first embodiment, the present disclosure provides stable, liquid, ready-to-use formulations of ketoprofen for parenteral administration, particularly in intravenous injection form for analgesic and/or anti-inflammatory use.

The formulations are ready to use, i.e., ready to be directly administered to a patient, without requiring a treatment step such as reconstitution or dilution. Therefore, these formulations offer an appreciable time savings for medical personnel, as well as the certainty of administering the correct medication formulation.

The formulations comprise ketoprofen (3-benzoyl-α-methylbenzeneacetic acid) in racemate form or one of its R or S isomers as the active ingredient. The active ingredient is not complexed, particularly with a compound such as piperazine or ethylenediamine. In some embodiments, the formulation comprises between 0.01 and 1% by weight of ketoprofen, particularly 0.1% by weight.

In some embodiments, the formulation comprises ketoprofen solubilized in a solely aqueous solvent to which a buffering agent has been added and the formulation is adjusted to a pH greater than 5.5 and less than 6.5.

The solely aqueous solvent mixture with the buffering agent constitutes a solubilization medium that has the function of putting the ketoprofen in liquid solution. A particular example of a solubilization medium contains water for injectable preparations and a buffering agent. The formulation is free of co-solvents, particularly non-aqueous solvents. In particular, it does not comprise a glycol or polyol such as polyethylene glycol or propylene glycol. It also does not comprise non-aqueous co-solvents of the alcohol type such as benzyl, ethanol or glycerol alcohol.

Advantageously, the solution is free of any other solubilizing agents such as surface active agents (polyoxypropylene/polyoxyethylene copolymer), arginine, N-methylglucamine, glycine, glucosamine, proline, hydroxyproline or alkyl ammonium compounds such as lysine. Consequently, in the formulations according to the present disclosure, the ketoprofen is not in complexed form or in the form of an organic salt, particularly nitrogen salt.

In fact, ketoprofen solubilization is ensured by the buffering agent. The buffering agent more or less maintains the same pH despite the addition of small quantities of an acid, base or dilution to the formulation. According to the present disclosure, the buffering agent may comprise at least one buffering agent selected from the group consisting of acetate, citrate, phosphate, malate, lactate and mixtures thereof. In certain embodiments, the buffering agent may be present in an amount of about 0.01 to about 2% by weight, particularly 1.2% by weight. This quantity varies depending on the nature of the buffering agent. Preferably, the buffer is acetate based, such as the acetate of an alkali salt or alkaline-earth salt, for example sodium acetate.

In general, the buffers are composed of a weak acid and its conjugate base. Advantageously, the buffer is an acetate/acetic acid pair, for example sodium acetate/acetic acid. According to a particular embodiment, the ratio between the acetic acid and the sodium acetate is between 1:20 and 1:500, particularly between 1:60 and 1:100.

The ketoprofen formulations according to the present disclosure present a pH of between 5.5 and 6.5, particularly between 5.8 and 6.3, for example 6. This pH is suitable for the intravenous injection of the formulation. The buffered formulation at this pH is stable, i.e., the ketoprofen does not degrade and its concentration is maintained substantially constant over time. In particular, the solution is stable for a period of over 3 months, advantageously over one year.

According to a particular embodiment, by choosing the acetate buffer and by adjusting the solution to a pH of around 6, the ketoprofen solution is stable. It does not require the addition of a preservative or antioxidant agent such as propyl gallate or sodium metabisulfite.

According to a particular embodiment, the formulations comprise at least one pH adjusting agent such as sodium hydroxide or hydrochloric acid to adjust the pH to around 6. In the case where the buffering agent is constituted of acetic acid/sodium acetate, using additional acetic acid as a pH adjuster is advantageous.

The parenteral administration of solutions requires that they be adjusted to isotony. The isotony of the composition may be obtained by adding an astutely calculated quantity of sodium chloride, glucose, mannitol, sorbitol, potassium chloride or calcium chloride, the often preferred isotonic agent being sodium chloride. The solution isotony is between 270 and 330 mOsm/kg, particularly between 280 and 290 mOsm/kg.

The acetic acid/sodium acetate buffering agent enables not only a solution buffered to a pH approaching 6 to be obtained, but also an isotony close to that of plasma to be obtained. In this case, it is not necessary to add an isotonic agent.

According to a particular embodiment of the invention, the formulations do not comprise an isotonic agent such as glucose or sodium chloride.

In another embodiment, the present disclosure provides a method for preparing ketoprofen formulations comprising dissolving ketoprofen in a solely aqueous solvent comprising a buffering agent, and adjusting the pH of the composition to a value of about 5.5 to about 6.5.

According to an embodiment, a formulation is prepared from the ketoprofen base, i.e., in the form of a free acid.

The pKa of ketoprofen is on the order of 4.6. The ready-to-use formulation has a pH of between 5.5 and 6.5. Consequently, the ketoprofen in the formulation is partly salified.

When the buffer is based on an alkali salt of acetate, the alkali salt of ketoprofen is formed. In the final formulation, the ketoprofen is present in salt and base form.

When the buffer is an acetic acid and acetate based pair, the preparation method comprises dissolving the ketoprofen in water containing acetate, and adding acetic acid to obtain a stable aqueous solution of ketoprofen and to adjust the pH to around 6.

During the first step, the solution comprising water and the buffer base is basic, enabling the ketoprofen to dissolve. With the addition of the ketoprofen and buffer acid, the solution becomes acid and stabilizes. After the formulation constituents are added and the pH is adjusted, the formulation may be sterilized by sterilizing filtration or heating.

The formulation may then be packaged in flexible bags or flasks away from light. For example, the bags may be made of polyethylene, polypropylene or polyvinyl chloride.

According to another aspect, the present disclosure relates to a sterile bag comprising a stable, liquid, ready-to-use formulation of ketoprofen.

In order to protect the formulation from light, the bags containing ketoprofen are overwrapped in a packaging that is at least partially opaque to light, such as for example, aluminum bags or a package constituted of 90% aluminum and 10% UV-impermeable plastic. In another example, the packaging is in the form of a bag comprising an opaque aluminum wall and a transparent plastic wall. In a variation, the opaque packaging comprises a non-opaque window.

According to a particular embodiment, the thickness of the aluminum film constituting the overwrap is between 5 and 25 μm, particularly 9 and 20 μm.

Advantageously, the ketoprofen is also protected from light during its production. For example, during production, the bags are protected in a UV-impermeable overwrapping.

According to one embodiment, the ketoprofen formulation comprises another analgesic and/or anti-inflammatory compound, such as for example paracetamol. In this case, as paracetamol is very sensitive to oxygen, the formulation particularly contains an oxygen content of less than 0.2 ppm, particularly less than 0.1 ppm, to prevent degradation of the paracetamol. To do this, the formulation undergoes, for example, sparging by using an inert gas such as nitrogen or treatment with pure steam. In addition, to prevent degradation of the paracetamol, having an oxygen scavenger between the bag containing the formulation and the overwrap is advantageous. The oxygen scavenger comprises, for example, an iron powder or iron oxide powder.

To facilitate a better understanding of the present invention, the following examples of certain aspects of some embodiments are given. In no way should the following examples be read to limit, or define, the entire scope of the invention.

### EXAMPLE

| | |
|---|---|
| Solution A | Solution B |

| | | | | |
|---|---|---|---|---|
| Ketoprofen | 100 | mg | 100 | mg |

| | | | |
|---|---|---|---|
| Paracetamol | — | 1 | g |

| | | | | |
|---|---|---|---|---|
| Glacial acetic acid | 14.2 | mg | 3.15 | mg |
| Sodium acetate trihydrate | 1910 | mg | 1490 | mg |
| (eq. anhydrous) | (1151 | mg) | (900 | mg) |

| | | |
|---|---|---|
| Sodium hydroxide | qs pH = 6 | qs pH = 6 |

| | | | | |
|---|---|---|---|---|
| WFI water QS | 100 | mL | 100 | mL |

Solution A is prepared as follows:
a) introducing ketoprofen and sodium acetate in powder form in 80 to 90% of the final volume of water for injectable preparations (WFI);
b) adding glacial acetic acid and adjusting the pH of the solution to around 6,
c) completing the final volume of the mixture with WFI water;
d) passing the ketoprofen solution through a sterilizing filter;
e) pouring the ketoprofen solution into a flexible bag, and
f) sealingly closing said bag in an overwrap that is opaque to light.

According to this formulation, the pH obtained is close to 6. It corresponds to an optimal pH for perfusion and for the stability of the ketoprofen in aqueous solution. In addition, this formulation presents an osmolarity of 287 mOsm/kg, which makes it suitable for intravenous administration without adding an isotonic agent.

Stability Study

Three lots of 50 L of solution A have been packaged in flexible 100-ml bags constituted from a polypropylene-based multilayer film. Each flexible bag is packaged in an aluminum bag. The lots have been placed in accelerated stability conditions, i.e., either at a temperature of +40° C. and a relative humidity of 20%, or at a temperature of +55° C. The following analyses have been carried out after 1 month, 2 months, 3 months: organoleptic characteristics, clarity, coloration, pH, ketoprofen assay, and ketoprofen impurities assay.

After three months of storage at +40° C. and 20% relative humidity or at +55° C., the samples of lots studied present good stability characteristics: The appearance of the solutions is unchanged and the clarity and coloration tests remain in conformance with the European Pharmacopoeia, the pH remains stable at 6 and the levels of impurities obtained do not increase with the temperature during preservation.

Consequently, this example demonstrates the stability of the ketoprofen formulation without the addition of a preservative or antioxidant.

The preparation of solution B is identical to that of solution A, with the additional step of introducing paracetamol in powder form in step a) of the preparation of solutions A.

The presence of paracetamol in the solution modifies the quantities of acetic acid and sodium acetate to obtain a pH close to 6. It should be noted that, without acetic acid, the pH of the solution is 6.11. The quantity of acetic acid to add to obtain a pH of 6 is thus minimal.

Under these conditions, the calculated osmolarity of the solution is 290 mOsm/Kg and does not require the addition of an isotonic agent.

To prevent oxidation of the paracetamol, the preparation of solutions containing paracetamol is done so as to maintain the quantity of oxygen dissolved in the solution below 0.2 ppm, advantageously below 0.1 ppm. To do this, the dissolved oxygen in the solution is eliminated for example by sparging in the solution of an inert gas such as nitrogen or by pure steam treatment, or a vacuum followed by the placement of a nitrogen blanket over the solution.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

What is claimed is:

1. A stable, liquid, ready-to-use ketoprofen composition comprising:
ketoprofen solubilized in a solely aqueous solvent comprising a buffering agent, wherein the composition has a pH of about 5.5 to about 6.5.

2. The ketoprofen composition of claim 1 wherein the ketoprofen is present in an amount of about 0.01% to about 1% by weight.

3. The ketoprofen composition of claim 1 wherein the ketoprofen is present in an amount of about 0.1%.

4. The ketoprofen composition of claim 1 wherein the buffering agent is present in an amount of about 0.01% to about 2% by weight.

5. The ketoprofen composition of claim 1 wherein the buffering agent is present in an amount of about 1.2% by weight.

6. The ketoprofen composition of claim 1 wherein the buffering agent comprises at least one buffering agent selected from the group consisting of acetate, citrate, phosphate, malate, lactate and mixtures thereof.

7. The ketoprofen composition of claim 1 wherein the buffering agent is acetic acid and sodium acetate.

8. The ketoprofen composition of claim 7 wherein the ratio by weight between acetic acid and sodium acetate is from about 1:20 to about 1:500.

9. The ketoprofen composition of claim 1 wherein the solely aqueous solvent is water for injectable preparations.

10. The ketoprofen composition of claim 1 further comprising at least one pH adjusting agent.

11. The ketoprofen composition of claim 1 wherein the pH of the composition is about 6.

12. The ketoprofen composition of claim 1 wherein the composition has an osmolarity of about 270 to about 330 mOsm/kg.

13. The ketoprofen composition of claim 1 wherein the composition is in the form of a formulation for intravenous injection.

14. The ketoprofen composition of claim 1 further comprising another analgesic and/or anti-inflammatory compound.

15. The ketoprofen composition of claim 14 wherein the analgesic compound is paracetamol.

16. A sterile bag comprising a stable, liquid, ready-to-use ketoprofen composition comprising:
ketoprofen solubilized in a solely aqueous solvent comprising a buffering agent, wherein the composition has a pH of about 5.5 to about 6.5.

17. A method of preparing a stable, liquid, ready-to-use ketoprofen composition comprising:
dissolving ketoprofen in a solely aqueous solvent comprising a buffering agent, and
adjusting the pH of the composition to a value of about 5.5 to about 6.5.

18. The method of claim 17 further comprising sterilizing the composition after the pH of the composition has been adjusted.
